(19) United States
(12) Patent Application Publication
(10) Pub. No.: US 2019/0000024 A1
(43) Pub. Date: Jan. 3, 2019

(54) Method for Breeding Hybrid Plants

(71) Applicant: University of Ljubljana, Ljubljana (SI)

(72) Inventors: Katarina Rudolf Pilih, Cerknica (SI); Jernej Jakse, Skofjica (SI); Borut Bohanec, Ljubljana (SI); Jana Murovec, Ljubljana (SI); Natasa Stajner, Ljubljana-Polje (SI)

(21) Appl. No.: 15/640,096

(22) Filed: Jun. 30, 2017

(51) Int. Cl. A01H 1/02; A01H 1/04; A01H 4/00

(52) U.S. Cl. CPC A01H 1/02; A01H 4/005; A01H 1/04

(57) ABSTRACT

The present invention relates to a method for breeding hybrid plants, comprising the steps of producing or providing essentially homozygous donor lines from a heterozygous starting population; genetically characterizing each donor line by means of molecular markers to obtain a genetic profile for each line; allowing the plants of the donor lines to intercross to obtain F1 hybrid progeny seed; sowing the F1 hybrid progeny seed while recording the maternal origin for each seed; phenotypically identifying superior F1 hybrid individuals among the progeny; determining the paternity of the superior F1 hybrid individuals for identification of their corresponding pollen donor lines; and crossing the thus identified pollen donor lines with female lines to obtain the hybrid plants.

### INTRODUCTION

### Field of the Invention

The present invention relates to a method for breeding hybrid plants, comprising simplified identification of inbred lines possessing superior combiner potential as parental lines for the hybrid plant.

### BACKGROUND

Plant breeding is one of the oldest achievements of mankind. The objective of plant breeding is to improve existing varieties and produce new ones that would suit the needs of farmers or consumers. Traditional plant breeding was done with an outbreak of civilizations by domestication of plants, by growing them under field conditions and by selecting those types that provided a useful source of food.

In general, selections are made from a collection of genetically diverse plants that can be derived from existing commercial varieties or gene bank accessions including old varieties, land races, wild relatives etc. From this collection, the “optimal” plants are selected and crossed according to the art. Plant breeding has the objective to produce improved crop varieties based on the exploitation of genetic variation, which exists within the germplasm of a plant species.

Improvement of varieties according to breeding goals is based on available genetic variation from described sources. In traditional plant breeding, the person skilled in the art selects groups of plants or individual plants that are supposed to possess genes or traits of interest. Further procedures are in some way related to the flowering characteristics of crops being either allogamous or autogamous, often termed “cross-pollinated” or “self-pollinated”. For cross-pollinated species, mass selection, with or without progeny testing, is perhaps the oldest of plant-breeding procedures resulting in a so-called “open-pollinated variety”, which is only partly homogeneous from a genetic and phenotypic standpoint. Open-pollinated varieties lack uniformity and hybrid vigour and therefore hybrid varieties are the preferred option. Breeding self-pollinated species tend to form pure lines (inbred lines) which are phenotypically uniform as a result of the mechanism of self-pollination. However, also these inbred lines lack hybrid vigour. Although in autogamous species pure-line breeding was so far a predominant method, lately hybrid cultivars tend to get a major role due to their advanced characteristics and faster combinations of valuable traits.

Hybrid vigor has been demonstrated in the early 20th century after hybrid corn was invented. These discoveries lead to high yield increases in all major crops tested. Hybrids are preferred varietal forms since they can provide better yield, greater uniformity and faster identification of desired combinations of characters. Hybrid varieties are also preferred by breeding companies since the progeny of the next F2 generation from those F1 hybrids will segregate and therefore not consistently express the desired characteristics. Hybrid varieties are now available for instance in crops such as maize, rice, wheat, barley, rye, sorghum, sugar beet, sunflower, beans, castor beans, oilseed rape, leek, onion, cucumber, tomato, spinach, melon, pumpkins, pepper, carrot, cabbage, cauliflower, broccoli, Chinese cabbage, radish, egg plant, hemp, cyclamen and lilies.

Such varieties are most often based on the crossing of two true breeding lines that may genetically complement each other. Often this complementarity of genotypes of genetically different parental lines, in the F1 hybrid results in a considerable improvement of e.g. growth characteristics, yield or adaptation to environmental stresses as compared to the individual parental lines and non-hybrid cultivars. Such enhancement of yield or strength is generally referred to as heterosis or hybrid vigour. Another term that relates to heterosis is “combining ability”. Combining ability is the phenomenon that only some inbred lines, when crossed to each other, complement each other in desired traits or enhance some traits. Also the opposite—bad combining ability—may result in an F1 hybrid that is not suitable or better than either of the individual parental lines, expressing negative heterosis or a lack of heterosis. Traditionally, breeders perform test crosses between putative parental lines to investigate the performance of the resulting offspring.

The production of hybrid varieties using traditional methods is however rather complicated, laborious and long lasting. Briefly, it typically consists of the following steps. To produce an F1 hybrid variety, several putative parental lines obtained from heterozygous sources are first made homozygous by several generations of inbreeding from a genetically heterogeneous genepool. Lately, haploid induction followed by chromosome doubling often replaces inbreeding and is now recognized as the most convenient method to produce inbred lines. The invention of doubled haploid techniques either by regeneration of plants derived from haploid egg cells (gynogenesis) or regeneration of plants derived from microspores (androgenesis) improves production of inbred lines, the process is faster than selfing and generates completely homozygous plants. Such plants are generally haploid, unless spontaneous diploidisation had occurred during the procedure. Chemical compounds that interfere with mitosis, such as colchicine, may be used to double the genome of haploid plants. Plants derived from such doubled haploid technology are completely homozygous and breed true. These methods are for instance described in detail by Murovec and Bohanec (Haploids and Doubled Haploids in Plant Breeding. In: Plant breeding. Edited by I. Y. Abdurakhmonov. INTECH, (2012)). Alternatively, non-reduced gametes obtained by second division restitution can be used to produce near inbred lines (EP-2301326).

The next step in hybrid breeding is identification of the best combination of previously obtained inbred lines. This step represents the major limitation of F1 hybrid breeding since an excessive amount of work is needed for testing of combining abilities. The aim is to identify two suitable lines that complement each other as explained above. However, in most breeding programs it is almost impossible to test combining ability of each line to each other due to excessive testing needed. Plant breeders often tend to produce large number of inbred lines in a breeding cycle, for instance one thousand or more being a very common number. To perform all possible combinations a series of crosses required is n² with reciprocals or n(n−1)/2 without reciprocals (n being the number of inbred lines). Testing combining ability of each of a thousand lines would mean one million reciprocal or 499,500 non-reciprocal crosses. For this reason testing for combining ability is usually done in two steps, the first step being testing for general combining ability. For this, all inbred lines are crossed with one or more testers and progeny is than analyzed to select lines with the highest general combining ability. This general combining ability test is done to avoid testing of combining ability of each line to all others, but does not give an exact value of the tested lines, since complementation of individual lines is extremely difficult to predict.

In traditional F1 hybrid breeding, the next step is testing for specific combining ability. In this step, lines with putatively high general combining ability are crossed with each other. This is done in one or in both directions (one-way or reciprocal crossing scheme).

When lines possessing desired traits, when combined in F1 hybrid, are identified, additional efforts are often made to obtain hybrid seeds in adequate quantities to satisfy market demand.

Testing inbred lines for their combining ability is the most limiting factor in an F1 hybrid breeding procedure.

### SUMMARY

It is therefore the object of the present invention to provide methods by which a much higher number of line to line tests can be performed.

This is achieved by a method for breeding hybrid plants, comprising:

a) producing essentially homozygous donor lines from a heterozygous starting population;

b) genetically characterizing each donor line by means of molecular markers to obtain a genetic profile for each line:

c) allowing the plants of the donor lines to intercross to obtain F1 hybrid progeny seed;

d) sowing the F1 hybrid progeny seed while recording the maternal origin for each seed;

e) phenotypically identifying superior F1 hybrid individuals among the progeny;

f) determining the paternity of the superior F1 hybrid individuals for identification of their corresponding pollen donor lines; and

g) crossing the thus identified pollen donor lines with female lines to obtain the hybrid plants.

The method of the invention can replace the usual testing for general combining ability but can also be used in combination with it when general combining ability is used as a preliminary test.

The essentially homozygous donor lines in step a) are suitably produced by inbreeding, i.e. by self-pollination or mating among relatives, or by production of doubled haploids (DHs), such as by haploid induction, wide hybridization, gynogenesis or androgenesis.

In one embodiment, intercrossing of the lines is achieved by means of hand pollination with a pollen mixture or by a polycross method using natural means, such as wind or insects or similar means. In a polycross genotypes are planted together in specific form to ensure maximal pollination by all other lines.

In order to maintain the original maternal lines that are used in intercrossing for later use in the production of the hybrid plants, in one embodiment at least one flower per plant is protected from cross-pollination and selfed. Alternatively, each plant that is intercrossed can be maintained by vegetative multiplication. This way, the genetic constitution of the selfed or vegetatively multiplied progeny is identical to the original maternal lines, which can then be used as the maternal parent in the production of a desired hybrid. In one embodiment, vegetative multiplication comprises in vitro micropropagation or in vivo cloning.

The paternity of the superior F1 hybrid individuals is in one embodiment determined by comparing the genetic profiles of the superior F1 hybrid plants with the genetic profiles of the donor lines determined in step b), excluding alleles that originated from maternal donor lines from the superior F1 hybrid plant profiles and comparing the remaining alleles to the genetic profiles of the donor lines to identify the paternal donor lines of the superior F1 hybrid plant. The genetic profile of each donor line used in the method is determined by means of molecular markers and the results are suitably stored in a database. The progeny of the intercross contains alleles from both parents. By deducing the maternal alleles from the genetic profiles of the superior plants found in the progeny of the intercross, the paternal profile of these plants is obtained and with this profile the corresponding paternal parent of the superior hybrid can be found in the database of donor lines and used in the production of the superior hybrid plants.

The identification of superior individuals can be achieved by all means of phenotypic characterization including scoring of field performance, computer image phenomic analysis, metabolomic analysis and similar. It should be noted that genotypes produced by accidental self-pollination will not exhibit hybrid vigor and would thus not be selected.

In case a breeding program calls for testing of larger number of inbred lines this invention disclosed two other options. In the first option, larger numbers of potentially superior parental lines are tested in two or more cycles of the intercrossing/paternity testing method of the invention. In this way, the one or optionally multiple cycle(s) serve as a substitute for general combining ability testing but with much higher precision.

In the second option, a general combining ability test is performed as in a traditional hybrid breeding protocol but then selected lines enter the intercrossing/paternity testing method of the invention. In this case the difference to existing protocols lies in allowing a much higher number of inbred lines with putatively good combining ability to enter testing for specific combining ability.

The advantage of the present invention is illustrated as follows. For example, if a plant breeder wants to test specific combining abilities of 100 inbred lines using traditional approach he should perform 100², i.e. 10.000, hybridizations to obtain information about performance of all lines crossed to each other in a reciprocal way. In contrast, according to the present invention, only 100 pollinations of each line with a pollen mixture of all 100 inbred lines (intercrossing), is required to obtain the same number of combinations. This way each pollen grain can fertilize a separate ovule on the plant.

### BRIEF DESCRIPTION OF THE FIGURES

In this application, reference is made to the following figures:

FIG. 1: Method for combining ability testing of F1 hybrids by revealing paternal origin of superior individuals within intercrossed progeny: a single step procedure.

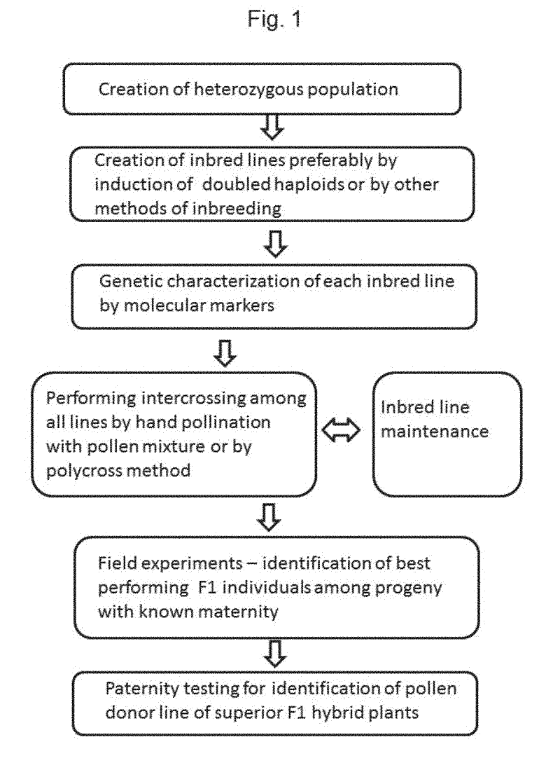

FIG. 2: Method for combining ability testing of F1 hybrids: Intercrossing of selected lines and paternity determination in a two (or more than two) step process. Lines are grouped and selected lines of each group enter next cycle of intercrossing. For details see FIG. 1.

FIG. 3: Method for combining ability testing of F1 hybrids: General combining ability test, intercrossing of selected lines and paternity determination. For details see FIG. 1.

FIG. 4: Scheme for discovery of paternal origin of superior F1 hybrid obtained by intercrossing with pollen mixture by co-dominant genetic marker system (Simple Sequence Repeats). The arrows refer to the characteristic combination of paternal alleles in inbred line (line 3) and F1 hybrid.

FIG. 5: Scheme for discovery of paternal origin of superior F1 hybrid obtained by intercrossing with pollen mixture by co-dominant genetic marker system (Single Nucleotide Polymorphism). The arrows refer to the characteristic combination of paternal alleles in inbred line (line 3) and F1 hybrid.

### DETAILED DESCRIPTION

The pollination with a pollen mixture or by the polycross method is an important feature of the invention that improves the efficiency of the method of the invention. This improvement lies in the fact that the combinations between the inbred lines is not one maternal line on one paternal line but that a batch of pollen is used for pollinating all maternal lines at once. In the traditional situation each flower of a plant of the maternal line is pollinated by pollen of the individual paternal plant. This leads to identical F1 progeny seed within one plant. In the situation of the invention pollination is random and each ovule in a flower within a plant is likely pollinated by a different pollen grain. One plant thus leads to a number of different progeny seeds.

In one embodiment, the genetic profiles of the donor lines and the superior hybrid progeny are determined by means of genetic markers. Over the last years, different genetic marker systems have been developed and applied to a range of crop species. Molecular markers are revealing polymorphisms at the DNA level and are now an important tool of modern genetics. However, there are various molecular biology techniques and procedures to produce them. Among those known to the person skilled in art are Restriction Fragment Length Polymorphisms (RFLPs), Random Amplified Polymorphic DNAs (RAPDs), Sequence Tagged Sites (STS), Amplified Fragment Length Polymorphisms (AFLPs). Simple Sequence Repeats (SSRs) or microsatellites, Single Nucleotide Polymorphisms (SNPs), direct sequencing of polymorphic DNA regions and others. One of possibilities well known attributed to molecular markers is their ability to discriminate among individual lines or hybrids due to their heterogeneous structure. Such approach is already used for instance for characterization of grapevine cultivars, where a set of 1.500 genotypes can be discriminated by combination of only nine SSR markers.

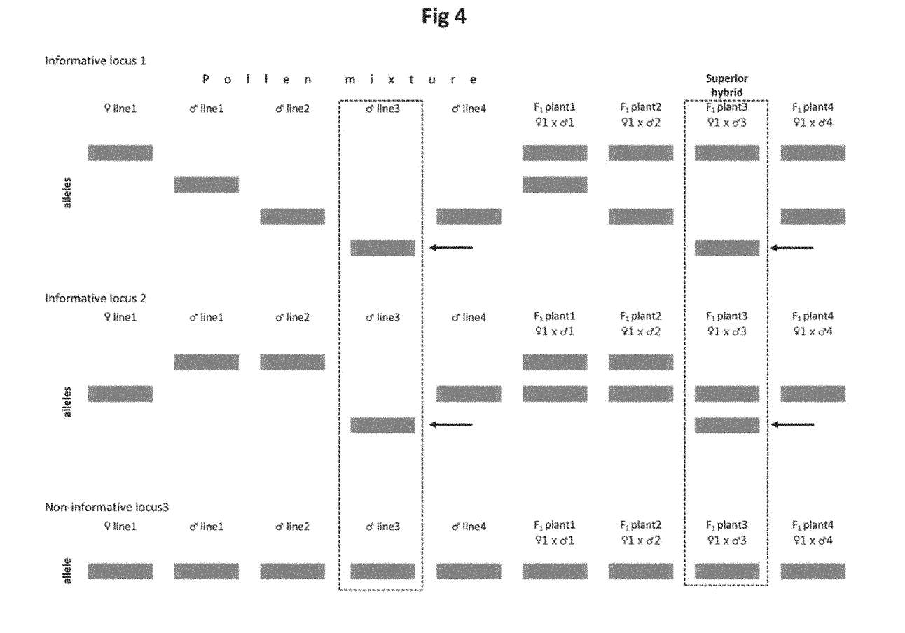

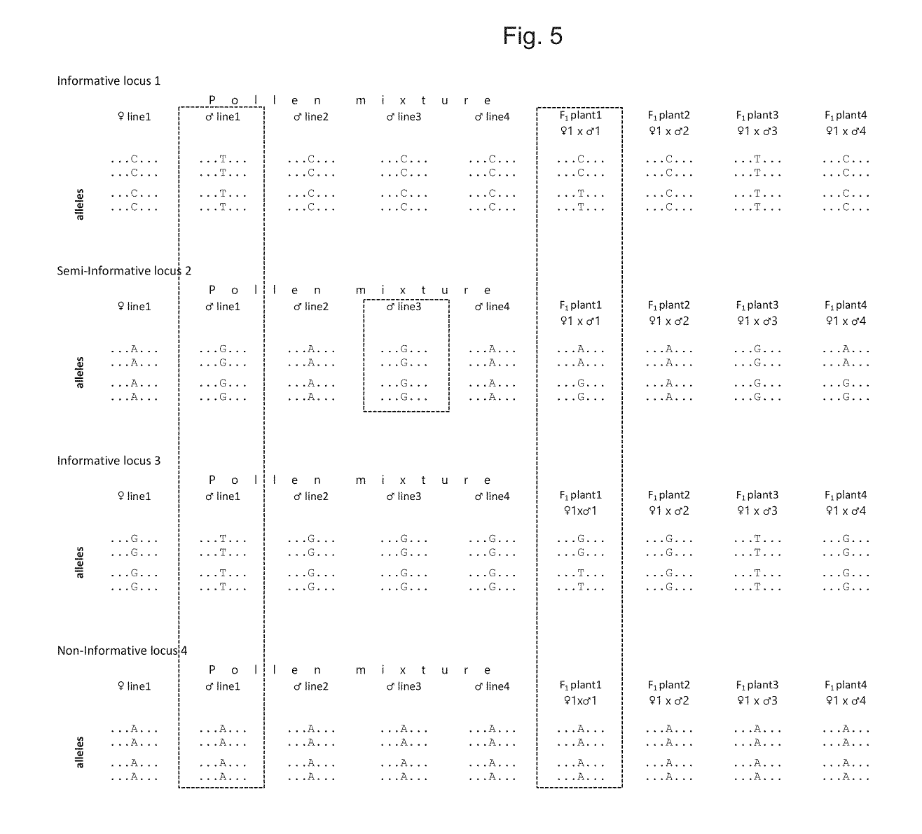

For the purpose of genetic characterization of inbred lines and hybrids almost all kind of genetic markers can be applied, however, marker systems based on “co-dominant” expression are preferred. For instance (but not exclusively), a typical choice of markers would be multi allelic simple sequence repeats (SSR) (FIG. 4), single nucleotide polymorphism (SNP) (FIG. 5) or analysis of polymorphic DNA sequence. By the use of these markers each individual plant can be described by its unique genetic profile. This would further lead to the database of distinguishable genetically profiled inbred lines as used in this invention.

Focusing on SSR studies for genotyping studies are mostly accomplished using PCR and capillary electrophoresis approach (CE) based on identification of length polymorphisms. The main challenge when using this methodology is the standardization of the allele sizes when comparing two or more data-sets. At this step, manual sizing and editing is required, which must be very precise to avoid false sizing. Besides, data analysed by CE technique, does not allow the determination of a full sequence of microsatellites but limits the information to the length polymorphism, which also hinders straightforward comparison of the data sets. As an alternative. Next Generation Sequencing methods (NGS) offer information about DNA sequences including identification of sequence variants of microsatellite loci and of their flanking regions. This advanced approach could provide a deeper insight and more precise evaluation of allele variants applicable for sample identification and paternity analysis.

The NGS methodology is preferred also in a case of larger datasets as it allows high-multiplexed screening of up to 1000 samples in a single sequencing run and can be adapted for use on any next-generation sequencing platform. However, the original ligation based NGS strategy might be expensive and even time consuming and laborious when the number of different samples to be sequenced are high. For this reason, a ‘hybrid’ approach as disclosed by Bell et al. (BMC Genomics 15:1002 (2014)) is preferred, allowing to create a cost-effective amplicon library based on incorporation of barcode sequences into specific target primers (SSRs in the present case), while the sequencing platform specific adaptors are ligated in a subsequent reaction during library preparation.

The most ultimate molecular marker system is of course analysis of the DNA sequence itself and comparing variation in it among different individuals, which is becoming reality through NGS systems and markers sequencing approaches via methods like RAD-seq, GBS etc.

A SNP (single nucleotide polymorphism) marker is a single base change in a DNA sequence, with two possible nucleotides at a given position, since SNPs are usually biallelic due to the low frequency of nucleotide changes and due to a bias in mutations with transitions occurring more frequently than transversions. SNP utilization usually starts with SNP discovery if the markers are not yet known (sequencing of locus specific sequences, EST sequencing, RNA-seq sequencing, genomic sequencing). The next step is genotyping of SNPs where many techniques are available including direct hybridization methods (e.g. microarrays), restriction enzyme cutting, single strand DNA conformation and heteroduplexes, primer extension, oligonucleotide ligation assay, pyrosequencing, exonuclease detection (TaqMan), invader assay, etc. Many approaches for SNP detection are available as commercial kits.

Using molecular markers an individual plant with a characteristic genotype can be clearly identified. Molecular marker analysis enables also parental testing which is particularly used for paternity testing. Paternity analysis is used extensively in molecular evolution, molecular ecology and in forensic science. For such purpose some software applications were developed such as PATRI—paternity inference using genetic data (Signorovitch, J in Nielsen. R, 2002: http://people.binf.ku.dk/rasmus/webpage/patri.html). FAMOZ (Gerber et al., Mol Ecol Notes 3: 479-481 ((2003) (http://www.pierroton.inra.fr/genetics/labo/Software/Famozindex.html). CERVUS 3.0.7 (Kalinowski et al. 2007 http://www.fieldgenetics.com/pages/aboutCervus_New.jsp) or PARENTE 1.2 (Cercueil et al., 2002; http://www2.ujf-grenoble.fr/leca/membres:manel.html). The aim of this testing is to identify paternal identity. All these documents are incorporated herein by reference.

The idea of using paternity testing in plant breeding schemes is known in the prior art. However, in the known uses in these plant breeding schemes the parental lines entering polycross breeding are not inbred lines as in the present invention, but heterozygous selections. These heterozygous lines are clonally propagated for maintenance. Also, superior lines selected according to polycross performance are heterozygous thus being usually selected to construct a synthetic cultivar or a very heterogeneous F1 hybrid. For these reasons in the known paternity testing no individual homozygous lines or genotypes are selected in search for F1 hybrid performance as disclosed in the present invention.

As described above, F1 hybrid seed production is based on crossing of selected inbred lines. Several methods can be used to produce inbred lines. Of the methods of traditional inbreeding the one most often used is self-pollination which results in a faster approach towards homozygosity compared to the alternative method of full-sib mating. These traditional methods require several generations of selfing usually a minimum of five or more to obtain useful uniformity through homozygosity. Often, five generations means five or more years, depending on the species. Alternatively, this traditional method of selfing has been improved by procedures allowing a more rapid flowering/embryo formation. Such fast generation cycling has been described in several plant species, for instance for legumes, in wheat, oat, triticale, and rice. Using fast generation cycling a higher degree of homozygosity is obtained in a shorter period of time, but the inbred lines that are obtained are still partially heterozygous.

Optimal methods for inbred line development from heterozygous donor lines are protocols based on production of haploid plants. Compared to other methods, induction of haploid plants from gametic tissues followed by chromosome doubling provides a much faster option. Also obtained lines are completely homozygous, which is not the case with other methods of inbreeding. Methods typically build on the ability of male or female haploid cells to form an embryo or alternatively on the elimination of one set of chromosomes (so-called haploid induction). Multiple examples exist in the prior art for the production of DHs in various crops.

Haploid lines need to be converted back to diploid level by chromosome doubling. This doubling can occur spontaneously during the process of regeneration or is induced by various methods. Methods can involve, for example, treatment of the haploid cells with anti-microtubule substances such as colchicine, trifluralin, APM or others or by exposure to laughing gas (nitrous oxide). Information regarding these techniques is readily available to the skilled person.

Formation of doubled haploid lines to be used as the essentially homozygous donor lines in the method of the invention is a preferred method since the paternal origin of DH progeny can be recovered with a higher probability (up to 100%) than for partially heterozygous inbred lines obtained by selfing. In this latter case the number of polymorphic loci is higher.

For the step of intercrossing with hand pollination, the collection of pollen is needed. Several methods can be used for pollen collection and are mainly adjusted to floral characteristics of various species. Some usual techniques for pollen collection are described by Shivanna and Rangaswamy (Pollen Biology. A Laboratory Manual.: 5-7 (1992)) and more recently by COLOS honey bee research association (http://www.coloss.org/beebook/I/misc-methods/4/7/2 as 28 Dec. 2016) and by Volk (http://cropgenebank.sgrp.cgiar.or/images/file/procedures/collecting2011/Chapter25-2011.pdf, 2011). Also some mechanical methods of collecting pollen are known in the art. For example, U.S. Pat. No. 4,922,651 discloses an apparatus for effecting or improving pollination of plants. All these documents are incorporated herein by reference.

Once pollen is collected it is advisable to test for viability. Several methods exist to estimate pollen viability (Shivanna and Rangaswamy, A Laboratory Manual: 33-37 (1992b)). Traditionally pollen quality is determined by staining methods or by in vivo or in vitro pollen germination, each having its own characteristics with respect to reliability, analysis speed, and species dependency. A recent advanced methods for pollen viability estimation is based on their dielectric properties by impedance flow cytometry (IFC) (Heidmann et al., PLOS ONE Volume: 11 Issue: 11 (2016)). All these documents are incorporated herein by reference.

Following pollen collection and viability assessment, pollen can be immediately used for crossings or if preferred kept for a prolonged periods. Methods for pollen storage are well known in the art. Usually, as the first step before storage, pollen is dried and then put into vials that are stored at low temperatures. These low temperatures can differ, such as +4° C. in a fridge or typically at −20° C. to −80° C. in freezers. The pollen can also be cryopreserved in liquid nitrogen.

Preservation of pollen to be used in combining ability testing as described in this application allows breeders to perform pollinations of lines that do not flower simultaneously.

In some cases, depending on flowering type, emasculation at an appropriate developmental stage is performed prior to intercrossing to prevent excessive self-pollination. Manual emasculation can be replaced by other means such as the use of a gametocide, self-incompatibility, male sterility and other methods that prevent self-pollination. In monoecious plants such as maize or cucurbits bagging of female inflorescence prior to pollination can replace emasculation.

Various options exist to perform pollination. Pollination by collected pollen mixture can be done by hand or by spraying. For enhanced pollinations, some breeders use a mix of pollen with additive such as dry wheat or rice flour. Such typical case is described by Acar and Eti, New Zeal J Crop Hort Sci 36: 295-300, (2008). More detailed description of pollination techniques of various species has been elaborated by Ivančič (Hibridizacija pomembnejših rastlinskih vrst. Fakulteta za kmetijstvo p.p. 775 (2002)). All these documents are incorporated herein by reference.

Beside hand pollination one possibility to intercross among individual plants is to perform a polycross, in which hand pollination is replaced by wind or insects to provide random pollination. Traditionally, the polycross test is a method of genetic selection among clones or, alternatively, inbred lines that are being considered for the use in a synthetic cultivar. The polycross test provides means to perform random pollination among individual plants each of which should have equal opportunity to be pollinated by any of the others. The design is used in breeding to produce synthetic cultivars, for recombining selected entries of families in recurrent selection breeding programs, or for evaluating the general combining ability of entries. Several designs of distribution of individual plants within a polycross are in use, some of them being supported by computer application. Varghese et al. (J Appl Stat 42 (2015)), which is incorporated herein by reference, elaborated various options and provided computer application for simplified designing.

In case intercrossing is performed by the means of a polycross, the inbred lines used in the polycross test are preferably multiplied by cloning or by self-pollination of lines.

The next step in the method of the invention is the identification of superior individuals carrying a highly desirable set of allelic combinations. Such superior individuals can be found in the progeny of random crosses between inbred lines. This individual F1 hybrid plant, is heterozygous and unique and is identified by its phenotypic characteristics.

The traditional method of testing F1 hybrid vigour performance of inbred lines does not allow for the random hybridization among inbred lines by pollen mixture or by polycross method since in such case parental origin can only be attributed to maternal but not also to paternal line origin. The hybrids can then not be reconstituted because the paternal parent cannot be determined. Crossings done for the traditional methods for estimation of general and specific combining ability also result in obtaining not one but several genetically equal seeds of each combination which are then studied for their phenotypic characteristics. In the present invention only single genotypes obtained from individual seeds are characterized.

Testing individual plants for selected breeding traits can be done in the traditional manner by observing the phenotype but was lately improved by various image analysis methods usually called plant phenomics. Using specific software and computer image analysis individual plants are tested (among others) concerning development, water use, architecture, shapes and reflectance at a wide range of wavelengths, from visible light to heat imaging. Processes can be automated to make it possible to considerably accelerate the process of estimating the characteristics of a phenotype, to increase its accuracy, and to remove human caused subjectivism. This testing can be performed in both controlled and field conditions. In one embodiment, plant phenomic analysis can be used for the identification of superior F1 hybrid plants with an increased efficiency as compared to the traditional identification methods.

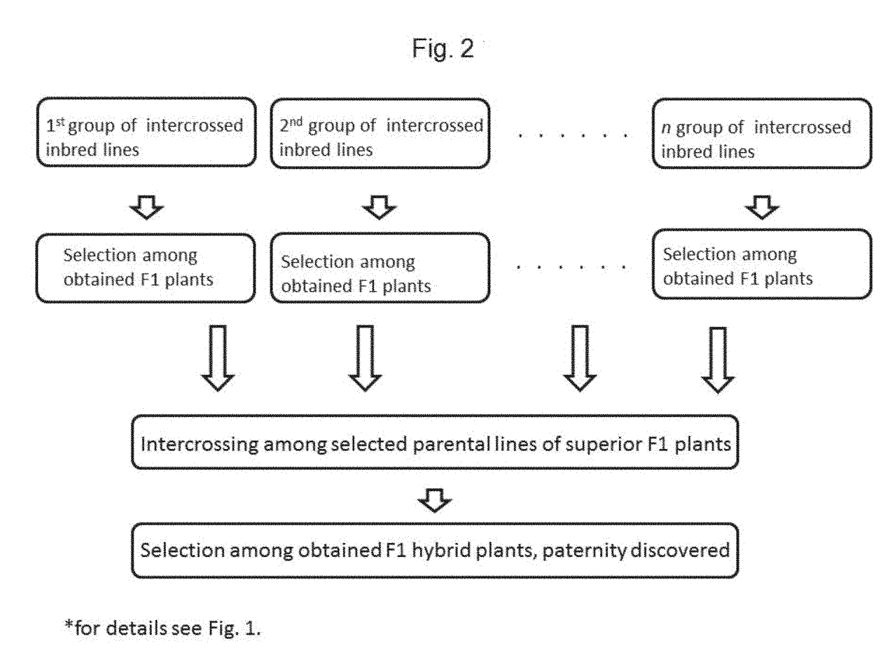

In case that a large number of lines are produced in a breeding program a single hand pollination with a pollen mixture or a single polycross might get physical limitations. In such a case not one but several separated polycrosses or hand pollinations with pollen mixture can be performed with different groups of inbred lines. Furthermore, the identified superior parental inbred lines (selected according to their F1 progeny performance) are then included in a second intercrossing scheme composed only of these superior lines (FIG. 2).

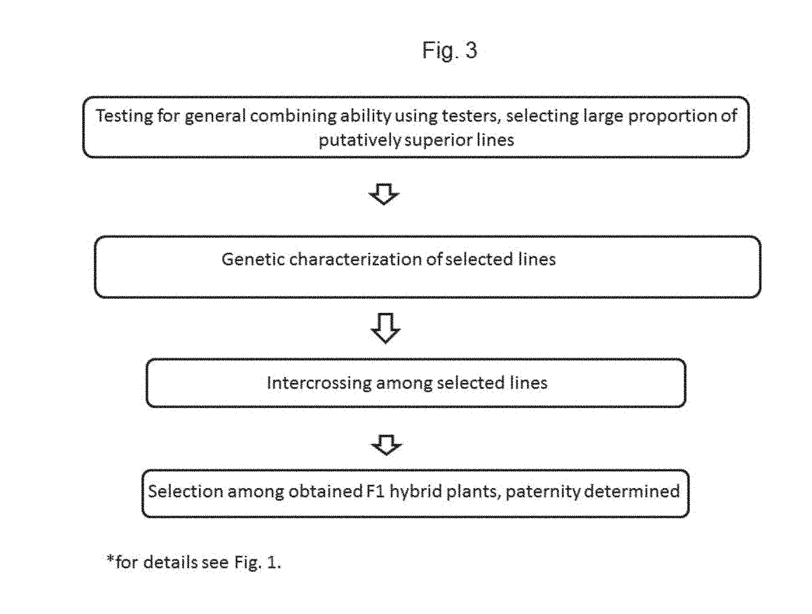

Alternatively, testing for combining ability can be performed according to standard methods using the known General combining ability test, but a much larger proportion of lines with putative positive combining ability can be selected than would be possible in a traditional protocol (FIG. 3).

The method of the present invention is applicable to a wide range of plants, in particularly plants that are sexually propagated, including, but not limited to: maize, rice, wheat, barley, rye, millet, pearl millet, sorghum, sugar beet, sunflower, cotton, beans, castor beans, oilseed rape, hemp, leek, garlic, onion, cucumber, tomato, egg plant, spinach, melon, pumpkins, pepper, carrot, cabbage, cauliflower, broccoli, Chinese cabbage, radish, cyclamen and lilies.

The present invention thus relates to the fields of plant improvement and plant breeding of all seed propagated plant species. The invention provides a method for breeding hybrid varieties by identification of inbred lines possessing superior combiner potential that results in hybrid vigour and comprises the steps of producing highly homozygous inbred lines, preferably DH lines obtained by the use of doubled haploid technique, from heterozygous parents and the maintenance of these lines, the genetic characterization of the said inbred lines by the use of molecular markers, preferably co-dominant molecular markers such as SSRs, SNPs, polymorphic sequences or any other means of nucleotide sequence analysis, intercrossing the lines by performing either a hand pollination with a pollen mixture or a polycross or any other pollination method to obtain maximal intercrossing, testing the F1 hybrid progeny of the intercrossed plants, of which the maternal origin has been recorded, for their phenotypic characteristics and identifying superior individual hybrid plants, determining the paternal line origin of the identified superior F1 plants by the analysis of their genetic profile while omitting mother plant alleles. In this way both parents of the superior hybrid can be identified in the maintained lines and be used to re-create the superior inbred lines.

As an alternative, the invention also provides protocols that comprise two or more cycles of said crossing, or the performance of the well-known General combining ability test prior to intercrossing according to the invention. Existing limitations in testing large number of combinations among inbred lines for heterotic potential are thus highly reduced.

Suitable techniques used in the various steps of the method of the invention will be illustrated in the Examples that follow and that are given for illustration purposes only and do not limit the invention in any way.

### EXAMPLES

### Example 1

### Induction of Haploid Cabbage Using Microspore Culture Technique

Buds of cabbage plants were harvested after the first three flowers in the inflorescences had fully opened. Each isolation of microspores was done with 64 flower buds ranging from 3.5 to 5.0 mm according to the genotype, microscopically determined to contain microspores at the late uninucleate stage. The buds were then sterilized in 16.7 g/l dichloroisocyanuric acid for 5 min and washed three times in sterile distilled water. They were then crushed in 1 ml of NLN media with 13% sucrose (Lichter, Z. Pflanzenphysiol. 103: 229-237 (1981)) hormone-free medium lacking potato extract at pH 6.0.

The microspore suspension released from the buds was filtered through 45 m nylon mesh. The residue on the nylon mesh was washed with 27 ml NLN medium and the filtrate was then transferred to four 10 ml centrifuge tubes and pelleted by centrifugation at 190 g for 3 min. The pellet was resuspended and washed three times with the same medium. After the final centrifugation, microspores from all four tubes were pooled to ensure equal representation in all treatments and then resuspended in NLN medium at a ratio of 1 bud/ml.

Desiccation and germination of embryos was initiated by treatment with abscisic acid (ABA). ABA was dissolved in 70% ethanol and added to individual Petri dishes. The culture medium was added after evaporation of the ethanol. Embryos were manually transferred to NLN medium containing 5 mg/l ABA and left on the shaker at 50 rpm at 25° C. in darkness. After 13 h the embryos were placed in 100 mm Petri dishes with one layer of filter paper (Whatman no. 40).

After 3 days any condensed water was removed by opening the lids for 10 min in a flow bench. This treatment resulted in drastic desiccation of embryos down to only 10-12% moisture. After 30-40 days desiccated embryos stored in darkness at 20° C. were placed on B5 medium (Gamborg et al., Exp. Cell Res. 50: 151-158 (1968)) containing 20 g/l sucrose at 20° C. for germination. Germinated embryos with the first two or three leaves developed were placed in 100 ml baby-food jars on the same medium. At the end of the subculture the plantlets were acclimatized in a greenhouse.

### Example 2

### Induction of Doubled Haploid Onion Plants Culturing Non-Pollinated Ovaries or Flower Buds

To induce haploid onion plants, donor onion plants are grown preferably in the greenhouse. At flowering, flower buds prior to dehiscence are collected and sterilized in 16.6 g/l dichloroisocyanuric acid disodium salt with the addition of a few drops of Tween 20 for 8 min. After three rinses in sterile water, the largest unopened flowers were selected and inoculated in 90-mm Petri dishes. Induction medium consisted of BDS macro, micro elements and vitamins (Dunstan and Short. Physiologia Plantarum 41: 1399-3054 (1977)), 500 mg/l inositol, 200 mg/l proline, 100 g/l sucrose, 7 g/l agar. pH 6.0, while hormones and sucrose levels differed. For the embryo induction medium, 2 mg/l 2,4-dichlorophenoxyacetic acid, 2 mg/l 6-benzylaminopurine and 100 g/l sucrose were added. Petri dishes were sealed with Parafilm and exposed to a 16/8 h photoperiod at 21-23° C. and illumination of 80 μmol m⁻²s⁻¹.

Flowers were left on this medium until the sprouting of embryos, which were subsequently transferred to elongation medium (induction medium supplemented with 60 g/l of sucrose). Embryos were treated for 2 days in liquid media supplemented with 50 μM APM to induce chromosome doubling. Plants were rooted in 150-mm test tubes on rooting medium (basal medium supplemented with 0.5 mg/l of indole-3-butyric acid and 40 g/l sucrose). Rooted onion plants were acclimatized in greenhouse conditions.

### Example 3

### Induction of Doubled Haploid Pumpkin Plants Using Irradiated Pollen

Pumpkin plants were grown in spring and summer in greenhouse and open-field conditions managed using standard agronomic practices. Male and female flowers were isolated 1 day before opening. The next morning anthers were collected, placed in Petri dishes, and irradiated at 200 Gy using X-rays. Female flowers were pollinated immediately after irradiation (from 6.00 to 10.30 hr) and re-isolated. In vitro embryo culture was performed. Immature fruit were harvested about 4 weeks after pollination and cleaned under tap water. Seeds were extracted, surface-sterilized for 20 min using dichloroisocyanuric acid sodium salt in a 2% solution (w/v) with Tween 20 added as a surfactant, washed with sterilized water over a sterile stainless steel mesh, and opened aseptically in a laminar flow hood. The excised embryos were cultured on solid E20A medium in 100-mm square petri dishes with 25 compartments at 23° C. with a 16-h photoperiod.

### Example 4

### Induction of Doubled Haploids of Winter and Spring Barley, Using Anther Culture

Tillers of barley were collected when the majority of microspores were at mid- and late-uninucleate stage. The developmental stage of microspores was checked in anthers from flowers located in the middle of the spikes, using a microscope. Tillers with spikes at the desired stage were wrapped in cellulose foil (Tomofan, Poland) and stored in Erlenmeyer flasks with tap water, in the dark at 4° C. for 4 weeks. Spikes were surface-sterilized in 70% ethanol for 1 minute and then in 10/o sodium hypochlorite for 20 min and rinse five times with sterile water. Anthers were aseptically excised and placed in Petri dishes with the N6L induction medium containing macro- and microelements according to Chu (Proc. Symp. Plant Tissue Cult., Beijing pp. 43-50 (1978)) with modifications. Cultures were grown in the dark at 26° C. After 3-4 weeks, the first embryo-like structures (ELS) were observed. For the following 3-4 weeks, ELS of about 1 mm in diameter were successively transferred onto the regeneration medium K4NB (Kumlehn et al. Plant Biotechnol. J. 4, 251-261 (2006)) with modifications. The ELS cultures were kept under light with 16-h photoperiod at 26° C. for about 2 weeks.

Developing plants were transferred to flasks with the N6I rooting medium containing macro- and microelements according to Chu (1981, supra) supplemented with additional components. For the preparation of media, one volume of double-concentrated solution was filter-sterilized and mixed with one volume of adequately concentrated Phytagel, which had been autoclaved with the respective proportion of distilled water. The pH was adjusted prior to filter sterilization of the solutions. Green plants with well developed roots and shoots were potted into the soil.

Measurements of the ploidy levels of plants were evaluated by fluorescence 40-60-diamidino-2-phenylindole (DAPI) using Partec 11 (Germany) flow cytometry. On the basis of collected data, the overall efficiency of DH plants production was calculated, as the number of green DH plants per 100 plated anthers.

### Example 5

### SSR Markers: PCR Amplification and Detection by Capillary Electrophoresis

SSR or microsatellite markers are widely used codominant markers and were developed for variety of plant species. Nowadays, these markers exist for most common crop or horticulture species. If they do not yet exist they can be easily developed from traditional (Brady et al., Euphytica 91, 277-84 (1996)), enriched genomic libraries (Jake & Javornik, Plant Mol. Biol. Rep. 19: 217-26 (2001)) or from available next generation sequencing (NGS) data (Zalapa et al., American Journal of Botany 99, 193-208 (2012)).

For illustrative description of the genotyping SSR analysis (Radosavljević et al., American Journal of Botany 98, e316-e8 (2011)) of the sage plant (Salvia officinalis L.) was taken as an example. Any other plant's SSR genotyping procedure is very similar.

PCR: Amplification with genomic DNA (10 ng) and reaction mixture (1×PCR buffer, 1.5 mM MgCl₂, 0.2 mM of each dNTP, 0.5 μM of each primer (given in Table below) where one of the primers is fluorescently labelled, 1 unit of Taq polymerase) is performed by a two-step PCR protocol with an initial touchdown cycle. The cycling conditions are as follows: 94° C. for 5 min; five cycles of 45 s at 94° C., 30 s at 60° C., which was lowered by 1° C. in each cycle, and 90 s at 72° C.; 25 cycles of 45 s at 94° C., 30 s at 55° C., and 90 s at 72° C.; and an 8-min extension step at 72° C. Samples are kept at 4° C. until analysis.

| Primer | |
|---|---|
| name | Primer sequence (5′-3′) |
| SoUZ012 | F: ACCATTGGAAAGATGCCTCA (SEQ ID NO: 01) |
| | R: GAATGGAGCGAGGAAGAAGA (SEQ ID NO: 02) |
| SoUZ013 | F: ACCATGCCCAAAGACCATAA (SEQ ID NO: 03) |
| | R: GGCTTCTCCCCTCGAATAAC (SEQ ID NO: 04) |
| SoUZ014 | F: GGCAATGATAAGGATGCTG (SEQ ID NO: 05) |
| | R: GAAGCTTCTCCCTTCTCTCTAACA (SEQ ID NO: 06) |
| SoUZ015 | F: CCATGTGTGAGTGTGTTGACC (SEQ ID NO: 07) |
| | R: ATCCAATTCGATTTGTTTACACC (SEQ ID NO: 08) |
| SoUZ016 | F: GGCGTTGCAGAGAGAGTGA (SEQ ID NO: 09) |
| | R: GGGCCTAGCCCTTTCTCTAT (SEQ ID NO: 10) |
| SoUZ017 | F: ACACCGACTCCATGCTGTAA (SEQ ID NO: 11) |
| | R: CCGGCACTCCCTCTATTTC (SEQ ID NO: 12) |
| SoUZ018 | F: CCATGTCAAGCTTCAAGAGGA (SEQ ID NO: 13) |
| | R: TTCATGCAACACATCCTTGA (SEQ ID NO: 14) |
| SoUZ019 | F: CAAAGCTCCTCGAAGACGAA (SEQ ID NO: 15) |
| | R: CACGAGCAAGCGTAATAGCA (SEQ ID NO: 16) |
| SoUZ020 | F: CCGGTTTCGAGAATTTGAG (SEQ ID NO: 17) |
| | R: AGCCCTGCAAATCCACTCTA (SEQ ID NO: 18) |

### Capillary Electrophoresis

The PCR products are mixed with the same volume of deonized formamide and appropriate size standard (e.g. GeneScan 600LIZ), heat denatured, chilled on ice and run on a capillary electrophoresis system ABI 3730XL analyzer (Applied Biosystems) or similar following the recommended procedure. Resulting electropherograms are analyzed using GeneMapper 4.0 software (Applied Biosystems) or PeakScanner (Applied Biosystems).

### Example 6

### GBS Markers

Next-generation sequencing (NGS) technologies have been recently used for whole genome sequencing and for re-sequencing projects where the genomes of several specimens are sequenced to discover large numbers of single nucleotide polymorphisms (SNPs) for exploring within-species diversity, constructing haplotype maps and performing genome-wide association studies (GWAS). Genotyping-by-sequencing (GBS) approach which relies on genome complexity reduction is suitable for population studies, germplasm characterization, breeding, and trait mapping in diverse organisms. The procedure can be generalized to any species and is based on high-throughput, next-generation sequencing of genomic subsets targeted by restriction enzymes.

Species selection of restriction enzymes (Res) that leave 2 to 3 bp overhangs and do not cut frequently in the major repetitive fraction of the investigated genome is very important. A suitable restriction enzyme for maize for example is ApeKI which creates a 5′ overhang (3 bp) and is partially methylation sensitive.

The sequences of the two oligonucleotides comprising the barcode adapter are: 5′-ACACTCTTTCCCTACACGACGCTCTTCCGATCTxxxx (SEQ ID NO: 19) and 5′-CWGyyyyAGATCGGAAGAGCGTCGTGTAGGGAAAGAGTGT (SEQ ID NO:20), where “xxxx” and “yyyy” denote the barcode and barcode complement sequences. The second, or “common”, adapter has only an ApeKI-compatible sticky end: 5′-CWGAGATCGGAAGAGCGGTTCAGCAGGAATGCCGAG (SEQ ID NO:21) and 5′-CTCGGCATCCTGCTGAACCGCTCTTCCGATCT (SEQ ID NO:22). Barcoded adapters are prepared for many samples as needed and allow polling of the samples for NGS sequencing run.

Oligonucleotides pairs of each barcode adapter and a common adapter are mixed together in a 1:1 ratio, 0.06 pmol of the mix is aliquoted into a 96-well PCR plate and dried down. 100 ng DNA samples are added to individual adapter-containing wells and dried. Samples (DNA plus adapters) are digested for 2 h at 75° C. with ApeKI (New England Biolabs) in 20 ttL volumes containing 1 NEB Buffer 3 and 3.6 U ApeKI. Adapters are then ligated to sticky ends by adding 30 μL of a solution containing 1.66× ligase buffer with ATP and T4 ligase (640 cohesive end units) (New England Biolabs) to each well. Samples are incubated at 22° C. for 1 h and heated to 65° C. for 30 min to inactivate the T4 ligase. Sets of 48 or 96 digested DNA samples, each with a different barcode adapter are combined (5 μL each) and purified using a commercial spin column kit (QIAquick PCR Purification Kit; Qiagen) following manufacturer's instructions. Cleaned DNA samples are eluted in a final volume of 50 μL. Restriction fragments from each library are then amplified in 50 μL volumes containing 2 μL of pooled DNA fragments, lx Taq Master Mix (New England Biolabs), and 25 pmol, each, of the following primers:

(A)
(SEQ ID NO: 23)
5′-AATGATACGGCGACCACCGAGATCTACACTCTTTCCCTACACGAC
GCTCTTCCGATCT
and
(B)
(SEQ ID NO: 24)
5′-CAAGCAGAAGACGGCATACGAGATCGGTCTCGGCATTCCTGCTGA
ACCGCTCTTCCGATCT.

These primers have complementary sequences for amplifying restriction fragments with ligated adapters, binding PCR products to oligonucleotides that coat the Illumina sequencing flow cell and priming subsequent DNA sequencing reactions. Different adapterslprimers can be made for any other NGS platforms.

Cycling conditions are as follows: 72° C. for 5 min, 98° C. for 30 s followed by 18 cycles of 98° C. for 30 s, 65° C. for 30 s, 72° C. for 30 s with a final Taq extension step at 72° C. for 5 min. These amplified sample pools constitute a sequencing “library.” Libraries are column purified and checked by Agilent electrophoresis for evaluation of fragment sizes. Libraries are considered suitable for sequencing if adapter dimers (˜128 bp in length) are minimal or absent and the majority of other DNA fragments are between 170-350 bp. If adapter dimers are present in excess of 0.5%, libraries are constructed again using a few DNA samples and decreasing adapter amounts.

Single-end sequencing (86 bp reads) of one 48- or 96-plex library per Illumina's flow-cell channel is performed on Illumina's instrument or any other appropriate NGS system. Resulting sequences are either mapped to the available genome sequence or using appropriate bioinformatics pipeline where no genome information is available.

### Example 7

### Production of Hybrid White Cabbage Using the Method of the Invention

Several combinations of white cabbage genotypes of internal breeding lines of the Biotechnical Faculty of the University of Ljubljana and a few commercial hybrids (Burton F1 (Nickerson-Zwaan) and Atria F1 (Semenarna Ljubljana)) were intercrossed to create a heterozygous population and were used as donors of microspores. Plants were grown in one season to produce heads, which were later vernalized and induced to flower in the next growing season. Immature flower buds were collected and used for doubled haploid production via microspore culture as described in Example 1.

The plantlets thus obtained were tested for ploidy using flow cytometric measurements as described in Example 4. Only plants with spontaneously doubled chromosome number were used further. Following acclimatization, DNA of 347 plants was isolated and individual genotypes were determined by the use of SST markers as described in Example 5. For this purpose the following protocol was used.

Total genomic DNA was extracted from about 100 mg of individual plant leaf, using a common CTAB extraction method (Doyle & Doyle. Focus 12, 13-15 (1990)). Concentration was quantified by fluorimetry (Amersham Biosciences DyNAQuant 200) and DNA at a concentration of 5 ng/μl was used for PCR amplification.

PCR amplifications were performed in a total volume of 15 μl containing 15 ng DNA template, lx PCR reaction buffer, 3.0 mM MgCl₂, 0.8 mM of each dNTP, 0.45 unit Taq DNA polymerase; 0.15 μM of each primer (forward tailed primer and reverse primer). Each forward SSR primer has an 18 bp tail added (5′-TGT AAA ACG ACG GCC AGT-3) (SEQ ID NO:25) complementary to the M13 primer. Four different fluorescent dyes at a concentration of 0.2 μM (6-FAM (blue) and HEX (green). NED (orange), PET (red)) were used to label the M13 primer.

The SSR markers used are listed in the following Table.

| Marker (locus) | Sequences | Motif |
|---|---|---|
| BoESSR053-for | 5′-TTTGCCAAGAAGCCTGAAGT-3′ | (GAA)7 |
| | (SEQ ID NO: 26) | |
| BoESSR053-rev | 5′-TGTACCAGCTGCAACCTCTG-3′ | |
| | (SEQ ID NO: 27) | |
| BoESSR087-for | 5′-GTTTCCTCTTCCACCACCAA-3′ | (TCC)7 |
| | (SEQ ID NO: 28) | |
| BoESSR087-rev | 5′-AATCTATCAAGAGGGCCAAGG-3′ | |
| | (SEQ ID NO: 29) | |
| BoESSR338-for | 5′-TGTAGCCGAAAGGGAATGAG-3′ | (AC)10 |
| | (SEQ ID NO: 30) | |
| BoESSR338-rev | 5′-GTGCTTGCATCCAGAAACCT-3′ | |
| | (SEQ ID NO: 31) | |
| BoESSR391-for | 5′-GCGACCTGTTGAAGAAGGAG-3′ | (GAT)7 |
| | (SEQ ID NO: 32) | |
| BoESSR391-rev | 5′-TTCTCCGCAAGAAATACAAGG-3′ | |
| | (SEQ ID NO: 33) | |
| BoESSR484-for | 5′-ACCCATACGTCCACGTCAAT-3′ | (AGA)7 |
| | (SEQ ID NO: 34) | |
| BoESSR484-rev | 5′-GCAATCGTCTTTCCACCAAT-3′ | |
| | (SEQ ID NO: 35) | |
| BoESSR492-for | 5′-GCGCAGAATCCAGATCATAG-3′ | (GA)9 |
| | (SEQ ID NO: 36) | |
| BoESSR492-rev | 5′-GGCTGGAGTATGAGCGAGAC-3′ | |
| | (SEQ ID NO: 37) | |
| BoESSR632-for | 5′-CCCTGCAATTGAAAACCAGT-3′ | (TGT)7 |
| | (SEQ ID NO: 38) | |
| BoESSR632-rev | 5′-AAACCGTCCAAGGATCATCA-3′ | |
| | (SEQ ID NO: 39) | |
| BoESSR825-for | 5′-GGACAGCGACACATTGAGTG-3′ | (CCG)7 |
| | (SEQ ID NO: 40) | |
| BoESSR825-rev | 5′-GGGAAGAGGTTCCCAAACAT-3′ | |
| | (SEQ ID NO: 41) | |

The cycling conditions were as follows: 95° C. for 5 min; 10 cycles of 30 s at 95° C., 30 s at 65° C., which was lowered by 1° C. in each cycle, and 30 s at 72° C.; 25 cycles of 30 s at 95° C., 30 s at 55° C. and 30 s at 72° C.; and a 5-min extension step at 72° C. Samples were kept at 4° C. until analysis.

The PCR products were genotyped using Fragment Analyzer, an automated capillary electrophoresis system (ABI3130XL of Applied Biosystems). The genotyping results were analyzed with GeneMapper and genetic diversity was analyzed with GenAlEx (Peakall, R. and Smouse P. E. (2006) GENALEX 6: genetic analysis in Excel. Population genetic software for teaching and research. Molecular Ecology Notes. 6, 288-295). Based on similarity coefficients 30 and 36 genetically divergent plants per group were selected for further intercrossing. With the eight primers listed above it was possible to discriminate between all the lines included in each group.

Plants were vernalized and grown to maturity. At flowering stage, selfing and intercrossing were performed.

Two different intercrossings have been done using the following approaches. In the first method, plants were grown in the greenhouse. On three occasions within two weeks pollen was collected from all plants using flowers opened at the day of pollination. Pollen was mixed and applied to stigmas of existing opened flowers. In the second method, selected plants were placed in a cage in a greenhouse and bumblebees were added into the cage for pollination.

Two groups of genetically distant doubled haploid plants were selected consisting of 30 and 36 plants for the first and second method, respectively. On each plant one inflorescence was emasculated, self pollinated and bagged, the rest were left to be interpollinated. Seeds were formed by both pollination methods.

To confirm the ability to detect a pollen parent a selection of seeds obtained from individual plants (parents) of intercrossing trial with bumblebees were germinated in vitro on half strength B5 medium (Gamborg et al. Exp. Cell Res. 50: 151-158 (1968)). Total genomic DNA and PCR amplifications were performed as described above.

The DNA profiles of these plants (with female genotype known) were determined using the same eight SSR loci and subjected to paternity analysis. All analyzed SSR loci were polymorphic with the allele frequencies as follows:

| | No. of alleles | Allele size |
|---|---|---|
| Marker (locus) | per locus | range (bp) |
| BoESSR825 | 2 | 237-241 |
| BoESSR632 | 2 | 135-150 |
| BoESSR338 | 2 | 275-285 |
| BoESSR484 | 4 | 143-153 |
| BoESSR087 | 3 | 150-162 |
| BoESSR391 | 5 | 325-374 |
| BoESSR053 | 2 | 253-273 |
| BoESSR492 | 5 | 196-210 |

Using Cervus 3.0.7 software the following paternity identifications were revealed as follows: 35 out of 36 parental plants produced unique allelic pattern. Paternity of offspring being tested on 109 F1 plants was identified. Although mother plants in pollination cage were present without replications (therefore not following complete polycross scheme) diversity of male parents was high. With only three seeds per plant tested, 17 out of 36 available pollen parents were actually determined as male parents. Data are presented in the following Table:

| DH | Determined male parent of F1 progeny and | | | | | |
|---|---|---|---|---|---|---|
| plant No. | its trio LOD score* | | | | | |
| (mother | F1 plant 1 | | F1 plant 2 | | F1 plant 3 | |
| plants) | male p. | LOD | male p. | LOD | male p. | LOD |
| 1 | 346 | 10.20 | 76 | 11.30 | 192 | 10.70 |
| 11 | 48 | 9.81 | 53 | 10.90 | 275 | 13.80 |
| 28** | 59 | 8.59 | 59 | 8.59 | — | — |
| 40 | 275 | 12.60 | 1 | 11.50 | — | — |
| 43 | 121 | 11.10 | 276 | 8.35 | 53 | 10.90 |
| 48 | 240 | 8.35 | 272 | 9.18 | 346 | 9.55 |
| 52 | 249 | 9.22 | 311 | 18.40 | 243 | 12.70 |
| 53 | 11 | 10.90 | 79 | 11.00 | 249 | 9.48 |
| 59 | 240 | 8.79 | 346 | 9.60 | 240 | 8.79 |
| 65 | 121 | 12.20 | 121 | 12.20 | 43 | 9.85 |
| 76 | 1 | 11.30 | 275 | 13.70 | 275 | 13.70 |
| 79 | 48 | 9.31 | 261 | 10.60 | 53 | 11.00 |
| 99 | 275 | 13.10 | 240 | 10.70 | 59 | 9.33 |
| 104 | 311 | 14.50 | 236 | 12.20 | 236 | 12.20 |
| 105 | 275 | 13.90 | 275 | 13.90 | 275 | 13.90 |
| 121 | 311 | 17.20 | 43 | 11.10 | 65 | 12.20 |
| 181 | 240 | 10.80 | 43 | 9.42 | 311 | 16.20 |
| 189 | 275 | 13.30 | 275 | 13.30 | 272 | 11.30 |
| 192 | 192 | selfed | 275 | 12.50 | 1 | 10.70 |
| 198 | 236 | 13.60 | 236 | 13.60 | 240 | 10.60 |
| 210 | 311 | 14.00 | 249 | 8.58 | 104 | 8.99 |
| 236 | 346 | 12.00 | 240 | 11.10 | 342 | 12.70 |
| 240 | 11 | 10.80 | 59 | 8.79 | 198 | 10.60 |
| 243 | 272 | 13.70 | 275 | 12.90 | 1 | 11.20 |
| 249 | 65 | 10.50 | 276 | 9.68 | 275 | 12.50 |
| 261 | 79 | 10.60 | 181 | 10.60 | 198 | 11.00 |
| 265 | 240 | 11.10 | 40 | 11.20 | 240 | 11.10 |
| 272 | 346 | 11.40 | 240 | 10.50 | 1 | 13.20 |
| 274 | 104 | 11.80 | — | — | — | — |
| 275 | 105 | 13.90 | 261 | 12.30 | 240 | 11.10 |
| 276 | 249 | 9.68 | 249 | 9.68 | 276 | selfed |
| 281** | 240 | 8.59 | 261 | 9.72 | 261 | 9.72 |
| 311 | 121 | 17.20 | 198 | 14.60 | 65 | 15.90 |
| 341 | 249 | 10.40 | 261 | 10.20 | 261 | 10.20 |
| 342 | 261 | 11.40 | 236 | 12.70 | 240 | 10.30 |
| 346 | 240 | 10.30 | 346 | selfed | 240 | 10.00 |

*By convention a LOD score greater than 3.0 is considered evidence for linkage as it indicates 1000 to 1 odds that the linkage being observed did not occur by chance.
**These two mother plants with equal allelic pattern could be altered.

It is clearly demonstrated, that both methods of interpollination produce F1 hybrid seeds with known maternal origin, and that paternal identity can be revealed with high accuracy via marker analysis. It should be noted that the number of tested F1 plants subjected to paternity analysis in this example was much higher that will be actually needed in breeding, since when applied in breeding only few superior F1 plants with desired characteristics would be selected and subjected to paternity analysis.

1. A method for breeding hybrid plants, comprising:
a) producing essentially homozygous donor lines from a heterozygous starting population;
b) genetically characterizing each donor line by means of molecular markers to obtain a unique genetic profile for each line;
c) allowing the plants of the donor lines to intercross to obtain F1 hybrid progeny seed;
d) sowing the F1 hybrid progeny seed while recording the maternal origin for each seed;
e) phenotypically identifying superior F1 hybrid individuals among the progeny;
f) determining the paternity of the superior F1 hybrid individuals for identification of their corresponding pollen donor lines; and
g) crossing the thus identified pollen donor lines with female donor lines to obtain the hybrid plants.

2. The method of claim 1, wherein producing essentially homozygous donor lines in step a) is achieved by inbreeding or production of double haploids.

3. The method of claim 1, wherein intercrossing of the lines is achieved by means of hand pollination with a pollen mixture or by a polycross method using natural means, such as wind or insects.

4. The method of claim 1, wherein in each plant that is intercrossed at least one flower is protected from crosspollination and selfed for maintaining the line.

5. The method of claim 1, wherein each plant that is intercrossed is maintained by vegetative multiplication.

6. The method of claim 5, wherein vegetative multiplication comprises in vitro micropropagation or in vivo cloning.

7. The method of claim 1, wherein paternity of the superior F1 hybrid individuals is determined by comparing the genetic profiles of the superior F1 hybrid plants with the genetic profiles of the donor lines determined in step b), excluding alleles that originated from maternal donor lines from the superior F1 hybrid plant profiles and comparing the remaining alleles to the genetic profiles of the donor lines to identify the paternal donor lines of the superior F1 hybrid plant.

8. The method of claim 1, wherein steps a) to f) are performed in parallel on separate groups of lines to identify superior parental lines in each group.

9. The method of claim 8, wherein the identified superior parental lines are subjected to:
b) genetically characterizing each donor line by means of molecular markers to obtain a unique genetic profile for each line;
c) allowing the plants of the donor lines to intercross to obtain F1 hybrid progeny seed;
d) sowing the F1 hybrid progeny seed while recording the maternal origin for each seed;
e) phenotypically identifying superior F1 hybrid individuals among the progeny;
f) determining the paternity of the superior F1 hybrid individuals for identification of their corresponding pollen donor lines; and
g) crossing the thus identified pollen donor lines with female donor lines to obtain the hybrid plants.

10. The method of claim 1, wherein the essentially homozygous donor lines of step a) are identified by testing for general combining ability using tester plants.


SEQUENCE LISTING (41 sequences)

SEQ ID NO: 1 - DNA, 20 bp, Salvia officinalis
accattggaaagatgcctca

SEQ ID NO: 2 - DNA, 20 bp, Salvia officinalis
gaatggagcgaggaagaaga

SEQ ID NO: 3 - DNA, 20 bp, Salvia officinalis
accatgcccaaagaccataa

SEQ ID NO: 4 - DNA, 20 bp, Salvia officinalis
ggcttctcccctcgaataac

SEQ ID NO: 5 - DNA, 19 bp, Salvia officinalis
ggcaatgataaggatgctg

SEQ ID NO: 6 - DNA, 24 bp, Salvia officinalis
gaagcttctcccttctctctaaca

SEQ ID NO: 7 - DNA, 21 bp, Salvia officinalis
ccatgtgtgagtgtgttgacc

SEQ ID NO: 8 - DNA, 23 bp, Salvia officinalis
atccaattcgatttgtttacacc

SEQ ID NO: 9 - DNA, 19 bp, Salvia officinalis
ggcgttgcagagagagtga

SEQ ID NO: 10 - DNA, 20 bp, Salvia officinalis
gggcctagccctttctctat

SEQ ID NO: 11 - DNA, 20 bp, Salvia officinalis
acaccgactccatgctgtaa

SEQ ID NO: 12 - DNA, 19 bp, Salvia officinalis
ccggcactccctctatttc

SEQ ID NO: 13 - DNA, 21 bp, Salvia officinalis
ccatgtcaagcttcaagagga

SEQ ID NO: 14 - DNA, 20 bp, Salvia officinalis
ttcatgcaacacatccttga

SEQ ID NO: 15 - DNA, 20 bp, Salvia officinalis
caaagctcctcgaagacgaa

SEQ ID NO: 16 - DNA, 20 bp, Salvia officinalis
cacgagcaagcgtaatagca

SEQ ID NO: 17 - DNA, 19 bp, Salvia officinalis
ccggtttcgagaatttgag

SEQ ID NO: 18 - DNA, 20 bp, Salvia officinalis
agccctgcaaatccactcta

SEQ ID NO: 19 - DNA, 33 bp, Zea mays
acactctttccctacacgacgctcttccgatct

SEQ ID NO: 20 - DNA, 33 bp, Zea mays
agatcggaagagcgtcgtgtagggaaagagtgt

SEQ ID NO: 21 - DNA, 36 bp, Zea mays
cwgagatcggaagagcggttcagcaggaatgccgag

SEQ ID NO: 22 - DNA, 33 bp, Zea mays
ctcggcattcctgctgaaccgctcttccgatct

SEQ ID NO: 23 - DNA, 58 bp, Zea mays
aatgatacggcgaccaccgagatctacactctttccctacacgacgctcttccgatct

SEQ ID NO: 24 - DNA, 61 bp, Zea mays
caagcagaagacggcatacgagatcggtctcggcattcctgctgaaccgctcttccgatct

SEQ ID NO: 25 - DNA, 18 bp, Brassica oleracea
tgtaaaacgacggccagt

SEQ ID NO: 26 - DNA, 20 bp, Brassica oleracea
tttgccaagaagcctgaagt

SEQ ID NO: 27 - DNA, 20 bp, Brassica oleracea
tgtaccagctgcaacctctg

SEQ ID NO: 28 - DNA, 20 bp, Brassica oleracea
gtttcctcttccaccaccaa

SEQ ID NO: 29 - DNA, 21 bp, Brassica oleracea
aatctatcaagagggccaagg

SEQ ID NO: 30 - DNA, 20 bp, Brassica oleracea
tgtagccgaaagggaatgag

SEQ ID NO: 31 - DNA, 20 bp, Brassica oleracea
gtgcttgcatccagaaacct

SEQ ID NO: 32 - DNA, 20 bp, Brassica oleracea
gcgacctgttgaagaaggag

SEQ ID NO: 33 - DNA, 21 bp, Brassica oleracea
ttctccgcaagaaatacaagg

SEQ ID NO: 34 - DNA, 20 bp, Brassica oleracea
acccatacgtccacgtcaat

SEQ ID NO: 35 - DNA, 20 bp, Brassica oleracea
gcaatcgtctttccaccaat

SEQ ID NO: 36 - DNA, 20 bp, Brassica oleracea
gcgcagaatccagatcatag

SEQ ID NO: 37 - DNA, 20 bp, Brassica oleracea
ggctggagtatgagcgagac

SEQ ID NO: 38 - DNA, 20 bp, Brassica oleracea
ccctgcaattgaaaaccagt

SEQ ID NO: 39 - DNA, 20 bp, Brassica oleracea
aaaccgtccaaggatcatca

SEQ ID NO: 40 - DNA, 20 bp, Brassica oleracea
ggacagcgacacattgagtg

SEQ ID NO: 41 - DNA, 20 bp, Brassica oleracea
gggaagaggttcccaaacat